(19) United States
(12) Patent Application Publication
(10) Pub. No.: US 2025/0170345 A1
(43) Pub. Date: May 29, 2025

(54) NICOTINE CESSATION KIT AND STEPDOWN METHOD

(71) Applicant: NicQuit LLC, Chicago, IL (US)

(72) Inventor: Alexander Cook, Chicago, IL (US)

(21) Appl. No.: 18/963,256

(22) Filed: Nov. 27, 2024

Related U.S. Application Data
(60) Provisional application No. 63/603,595, filed on Nov. 28, 2023.

(51) Int. Cl. A61M 15/06; A61M 15/00

(52) U.S. Cl. CPC A61M 15/06; A61M 15/0001

(57) ABSTRACT

A kit and a method for nicotine cessation are disclosed. The kit includes a plurality of vessels each holding a substance for consumption by a user. The plurality of vessels includes: a first vessel with the substance having a highest nicotine content; a last vessel with the substance having a lowest nicotine content; a second vessel with the substance having a second nicotine content lower than the highest nicotine content; and at least a third vessel with the substance having a third nicotine content higher than the lowest nicotine content and lower than the second nicotine content. Nicotine content decreases gradually from the highest nicotine content to the lowest nicotine content and the substance held by the plurality of vessels is configured to be consumed in order from the first vessel to the last vessel.

### RELATED APPLICATION DATA

This application is entitled to the benefit of and claims priority to co-pending U.S. Provisional Application Ser. No. 63/603,595 filed Nov. 28, 2023 and entitled “Nicotine Cessation Kit Using Vapes That Follow a Nicotine Strength Stepdown Program.” The entire contents of this prior filed application are hereby incorporated herein by reference.

### BACKGROUND

### 1. Field of the Disclosure

The present disclosure is generally directed to products and methods designed to assist individuals in overcoming nicotine addiction, and more particularly to a nicotine cessation kit and a stepdown method using same.

### 2. Description of Related Art

The original purpose of e-cigarettes and vaping was to assist individuals in overcoming their dependence on smoking products, i.e., cigarettes, etc. Such dependence is typically caused by an addiction to nicotine contained within the products. However, it has become evident that many people have instead developed a dependence on vaping using e-cigarette products and substances.

Also, when users seek to overcome their nicotine habit or addiction while using e-cigarettes, a known technique is to reduce or halve their nicotine intake while using e-cigarettes. A typical e-cigarette with standard nicotine strength may have, for example, 24 milligrams (mg) of nicotine. This known technique involves a challenging, periodic, abrupt halving of nicotine levels, such as from 24 mg to 12 mg to 6 mg to 3 mg, etc., until reaching a nicotine-free dose of 0 mg. This abrupt reduction often results in heightened cravings, increased vaping frequency, and uncomfortable withdrawal symptoms.

### SUMMARY

In one example, according to the teachings of the present disclosure, a kit for nicotine cessation includes a plurality of vessels each holding a substance for consumption by a user. The plurality of vessels includes: a first vessel with the substance having a highest nicotine content; a last vessel with the substance having a lowest nicotine content; a second vessel with the substance having a second nicotine content lower than the highest nicotine content; and at least a third vessel with the substance having a third nicotine content higher than the lowest nicotine content and lower than the second nicotine content. Nicotine content decreases gradually from the highest nicotine content to the lowest nicotine content and the substance held by the plurality of vessels is configured to be consumed in order from the first vessel to the last vessel.

In one example, the substance can be an e-liquid configured to be consumed using an e-cigarette.

In one example, each of the plurality of vessels can be an e-cigarette filled with the substance. The substance can be an e-liquid configured to be consumed through vaping.

In one example, where each of the plurality of vessels is an e-cigarette, each e-cigarette can be a disposable e-cigarette pre-filled with e-liquid as the substance.

In one example, each of the plurality of vessels can be a fluid container holding the substance, which can bean e-liquid configured to be consumed through vaping.

In one example, the kit can include an e-cigarette provided as a part of the kit for vaping e-liquid as the substance, and each of the plurality of vessels can be a fluid container holding the e-liquid from which the e-cigarette can be filled.

In one example, the plurality of vessels can include unique indicia, which can inform the user which of the plurality of vessels is the first vessel, the second vessel, at least the third vessel, and the last vessel.

In one example, each vessel of the plurality of vessels can include a visible numerical indicia so that the plurality of vessels is sequentially numbered from the first vessel to the last vessel in the order to be consumed by the user.

In one example, each vessel of the plurality of vessels can be a nicotine pouch, a nicotine patch, or an oral consumable, and the substance can accordingly be consumed orally or through the user's skin.

In one example, the plurality of vessels can include at least ten vessels inclusive of the first, second, third, and last vessels.

In one example, the plurality of vessels can include exactly ten vessels. The ten vessels can be provided with sequential visible indicia as numbers from the first vessel bearing a ‘1’ to the last vessel bearing a ‘10’.

In one example, the lowest nicotine content of the last vessel can be in a range from 25% to 0% of an initial nicotine intake of the user.

In one example, the highest nicotine content of the first vessel can be in a range from 100% to 75% of an initial nicotine intake of the user.

In one example according to the teachings of the present disclosure, a method for nicotine cessation, includes acquiring a nicotine cessation kit having a plurality of vessels with a substance for consumption by a user. The plurality of vessels includes a first vessel with the substance having a highest nicotine content and includes a last vessel with the substance having a lowest nicotine content. The method also includes selecting a first vessel of the plurality of vessels with the substance having a highest nicotine content and consuming the substance of the first vessel. The method also includes selecting a next vessel of the plurality of vessels having a highest remaining nicotine content and consuming the substance of the next vessel. The method further includes repeating selecting a next remaining vessel and consuming the substance of the next remaining vessel until only the last vessel remains. The method also includes consuming the substance of the last vessel. Nicotine content decreases gradually from the highest nicotine content to the lowest nicotine content.

In one example, the substance can be an e-liquid and the steps of consuming can include vaping the e-liquid using an e-cigarette.

The method of claim 15, wherein each of the plurality of vessels is a disposable e-cigarette pre-filled with the e-liquid, and wherein the method includes discarding each disposable e-cigarette when the e-liquid therein has been consumed through vaping.

In one example, each of the plurality of vessels can be a container holding the e-liquid and the method can include filling the e-cigarette with the e-liquid from the containers and consuming the e-liquid through vaping.

In one example, the plurality of vessels includes sequentially numbered visible indicia. The steps of selecting can include identifying the first vessel by the visible indicia ‘1’, and identifying each next vessel in sequential numbered order to the last vessel.

In one example, each vessel of the plurality of vessels can be a nicotine pouch, a nicotine patch, or an oral consumable and the method can accordingly include consuming the substance orally or though the user's skin.

In one example, the plurality of vessels can include at least ten vessels inclusive of the first, each next, and the last vessels.

### BRIEF DESCRIPTION OF THE DRAWINGS

The drawings provided herewith illustrate one or more examples or embodiments of the disclosure and therefore should not be considered as limiting the scope of the disclosure. There may be other examples and embodiments that may be equally effective to achieve the objectives and that may fall within the scope of the disclosure. Objects, features, and advantages of the present invention should become apparent upon reading the following description in conjunction with the drawing figures.

FIG. 1 shows one example of a nicotine cessation system or kit including a series of disposable e-cigarettes according to the teachings of the present disclosure.

FIG. 2 shows one example of a nicotine cessation method or stepdown program using the series of disposable e-cigarettes of FIG. 1 according to the teachings of the present disclosure.

FIG. 3 shows one example of a nicotine cessation system or kit including a series of bottles of e-liquid according to the teachings of the present disclosure.

FIG. 4 shows one example of a nicotine cessation method or stepdown program using the series of bottles of FIG. 3 according to the teachings of the present disclosure.

The use of the same reference numbers or characters throughout the description and drawings indicates similar or identical components, aspects, and features of the disclosure.

### DETAILED DESCRIPTION OF THE DISCLOSURE

The present disclosure is directed to an innovative solution that solves or vastly improves upon the above noted and other problems with prior known techniques for overcoming nicotine addiction. The rise of vaping as a smoking cessation tool has reshaped the landscape of nicotine addiction and dependency, introducing both benefits and challenges. While initially intended for quitting smoking, vaping has led to a unique dependence on nicotine. Conventional cessation methods often involve abrupt reductions, resulting in heightened cravings and discomfort. The disclosed solution is a “Vape Quit Kit” and a stepdown method that improve the nicotine cessation process and address the above-noted and other problems with the prior known nicotine halving technique. The kits and methods provide a full range of nicotine strengths, guiding users through a program that makes it more achievable to quit vaping and overcome nicotine dependency.

When a component, device, kit, system, step, unit, module, element, or the like of the present disclosure is described as having a purpose or performing an operation, function, or the like, the component, device, kit, system, unit, module, element, or the like should be considered herein as being “configured to” meet that purpose or to perform that operation or function.

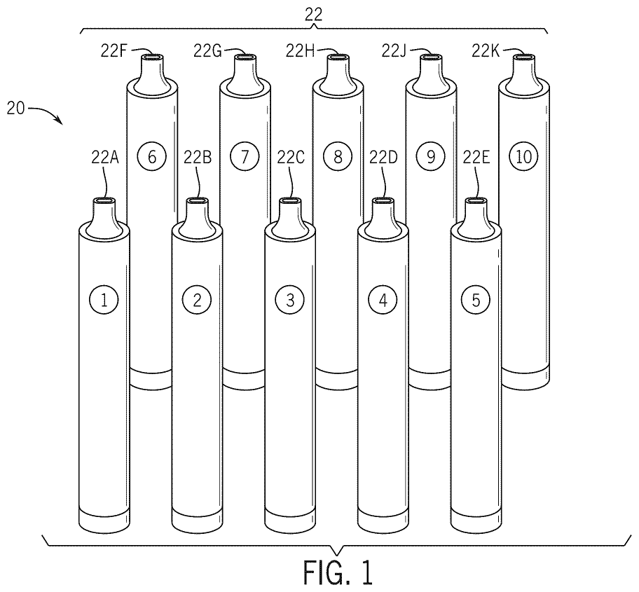

Various examples and embodiments of kits and methods for nicotine cessation are disclosed and described herein with reference to the drawings. FIG. 1 shows one example of a nicotine cessation kit 20, i.e., hereinafter the kit 20, that has been devised in accordance with the principles of the present disclosure. As should become evident upon reading the following description, the kit 20 may be modified in various ways without departing from the scope and spirit of the present disclosure.

The kit 20 in one example includes a plurality of vessels, where the plurality of vessels is generally identified as the vessels 22, and where the vessels 22 are configured to hold a substance that contains nicotine. In some examples, the vessels 22 may be in the form of nicotine pouches, i.e., lip pouches, which hold a substance that includes nicotine to be orally consumed, i.e., absorbed, through the soft tissue of the mouth of a user. In other examples, the vessels 22 may be in the form of nicotine patches, which hold a substance that includes nicotine to be consumed, i.e., absorbed, through the skin of a user. In still other examples, the vessels 22 may be in the form of nicotine lozenges, which hold a substance that includes nicotine to be orally consumed, i.e., absorbed, through the soft tissue of the mouth of a user. In yet other examples, the vessels 22 may be in the form of nicotine gum, which hold a substance that includes nicotine to be orally consumed, i.e., absorbed, through the soft tissue of the mouth of a user as the gum is chewed.

In the example of FIG. 1, the kit 20 includes vessels 22 that may be configured hold a substance, which is generally known as an e-liquid. In this example, the vessels 22 are also configured to produce and deliver the inhalable vapor yielded from the e-liquid to the user. Thus, in this example, the vessels 22 may be e-cigarettes or vape pens, which are generally known in the art. An e-cigarette is configured to heat the e-liquid so that the user can inhale the vapor. The configuration and construction of the vessels 22 in this example can vary in contour design, color, size, form, mouthpiece design, and/or vapor generating function without departing from the spirit and scope of the present disclosure. Each of the vessels 22 also includes a storage space or cavity (not shown) configured to hold a predetermined volume of the e-liquid. The amount of e-liquid that can be held by each of the vessels 22 can also vary depending on a number of variables that are discussed further below.

The e-liquid, which is also generally known in the art and which is also called, for example, vape juice or e-juice, is a liquid used in e-cigarettes or vape pens to create the inhalable vapor. The e-liquid typically is comprised of a mixture of a number of ingredients. For example, e-liquid typically includes propylene glycol (PG), which is a relatively thinner chemical that may effectively carry flavor and produce a relatively strong “hit” to the user's throat when inhaling the vapor. Further, e-liquid also typically includes vegetable glycerin (VG), which is a thicker chemical. Also, e-liquid very often includes nicotine, which is well known to be an addictive chemical substance. Nicotine may exist in the e-liquid at varying levels. Still further, e-liquid typically includes various flavoring chemicals designed to produce specific flavors that may be desired by a user and selectable from a variety of different flavors. Also, e-liquid typically includes chemicals that act as coolants designed to create a cooling sensation in the user's mouth. In some instances, e-liquid may also include water and/or other elements.

The ratio of PG to VG in an e-liquid can be adjusted to alter the viscosity of the e-liquid and, thus, for which type of delivery device, i.e., e-cigarette, the e-liquid may be best suited. As an example, a high VG content e-liquid ratio VG>PG, i.e., more VG than PG, has higher viscosity and may be better suited for more powerful and more expensive devices such as sub-ohm e-cigarettes. In contrast, an e-liquid having closer to a 1:1 PG to VG ratio or lower will have a relatively lower viscosity and be better suited for less expensive and less complicated or simpler delivery devices or e-cigarettes.

The present disclosure is concerned with e-liquids that contain nicotine and with helping users overcome their addiction to the substance. The present disclosure is not particularly concerned with the other chemicals and contents of e-liquids. The example in FIG. 1 of the present is also not limited to any particular type of delivery mechanism, device, or e-cigarette configuration. However, the vessels 22 of the kit 20 shown in FIG. 1 in one example may be disposable e-cigarettes that can be discarded as a user completes the steps in the disclosed processes or methods. Further, as described in more detail below, the plurality of vessels 22 may be pre-loaded or pre-filled with a specific or predetermined volume of e-liquid.

Referring again to FIG. 1, the plurality of vessels 22 in this example may include a predetermined number of e-cigarettes, such as e-cigarettes 22A-22K. As discussed further below, the number of vessels 22 in the kit 20 may vary according to the needs of a particular user and/or according to a particular kit implementation. In this particular example, the plurality of vessels 22 of the kit 20 includes ten (10) e-cigarettes 22A-22K.

In one example, each of the e-cigarettes 22A-22K may be preloaded or pre-filled with a predetermined volume of e-liquid. The volume of e-liquid preloaded into each of the e-cigarettes may vary, as discussed in further detail below. Also, the nicotine amount or content, i.e., strength, of each of the e-cigarettes 22A-22K in this example is different or varied in comparison to the remaining e-cigarettes of the kit 20. In other words, each e-cigarette 22A-22K of the plurality of vessels 22 is pre-filled with e-liquid having a predetermined nicotine strength. The strength or amount of nicotine is designed to gradually decrease from a first vessel or e-cigarette 22A, to among second through ninth vessels or e-cigarettes 22B-22J, and to a last or final vessel, i.e., a tenth e-cigarette 22K in this example. Thus, the first e-cigarette 22A in this example has the highest nicotine strength and the last or tenth e-cigarette 22K has the lowest nicotine strength.

In one example, the first e-cigarette 22A may be pre-filled with e-liquid having a highest nicotine strength that matches the nicotine strength of the vaping products currently used by a user, i.e., the initial nicotine intake of the user. In another example, the first e-cigarette 22A may be pre-filled with e-liquid having a highest nicotine strength that is slightly less than the nicotine strength of the user's current vaping products. The nicotine strength of the first e-cigarette 22A may be, for example, 5% less, 10% less, 15% less, 20%, or even 25% less than the nicotine strength of the user's current vaping products. For some user's, a 25% reduction from their current nicotine product strength to the first vessel may be too much to avoid or inhibit withdrawal symptoms or other negative responses. In one example, the highest nicotine strength or content of the first vessel, i.e., the first e-cigarette 22A, may be in a range of 5% to 20% of the user's initial nicotine intake. In yet another example, the first e-cigarette 22A may be pre-filled with e-liquid having a highest nicotine strength that is a designated or standard strength determined by the manufacturer or producer of the plurality of vessels 22. In still another example, the first e-cigarette 22A may be pre-filled with e-liquid having a highest nicotine strength that is predetermined by other means, such as an average strength among a predetermined or specific cross-section of users and vaping products.

In one example, the last e-cigarette 22K may be pre-filled with e-liquid having a lowest nicotine strength, which in one example is zero nicotine strength, i.e., the e-liquid has no nicotine content. In another example, the last e-cigarette 22K may be pre-filled with e-liquid having a lowest nicotine strength that is slightly more than zero or no nicotine. The nicotine strength of the last e-cigarette 22A may be, for example, 25%, 20%, 15%, 10%, or 5% of the highest nicotine strength of the first e-cigarette 22A of the kit 20 or of the user's initial nicotine intake. In one example, the nicotine strength of the last vessel, i.e., the last e-cigarette 22K, may be in a range of 20% to 5% of the highest nicotine strength or content or of the user's initial nicotine strength. In yet another example, the last e-cigarette 22K may be pre-filled with e-liquid having a lowest nicotine strength that is a designated or standard minimum or de minimis strength determined by the manufacturer or producer of the plurality of vessels 22. In still another example, the last e-cigarette 22K may be pre-filled with e-liquid having a lowest nicotine strength that is predetermined by other means, such as an amount of nicotine that is medically determined to be trivial or non-addictive.

The gradual reduction in nicotine strength from the first e-cigarette 22A to the last e-cigarette 22K of the kit 20 may be substantially or primarily consistent from first to last. In one example, the nicotine strength may be reduced by about 10% from the highest nicotine strength vessel or e-cigarette 22A, to the next highest lowest nicotine strength vessel or e-cigarette 22B, and so on to the last vessel or e-cigarette 22K of the kit 20. This strength differential among the vessels 22 may vary by 1% or 2% in this example, simply through manufacturing tolerance, as well as so that the decease may gradually reduce from highest to lowest among the ten (10) vessels 22. The graduation among the vessels does not need to be precisely the same but, in one example, may be relatively the same to achieve the intended goal of gradual reduction while avoiding the negative consequences of withdrawal, etc.

The highest nicotine strength of the first vessel 22A, the lowest nicotine strength of the last vessel 22K, and the and the number of vessels 22 in the kit will determine the gradual strength reduction from first to last among the vessels 22. The present disclosure is not limited to kits including ten (10) vessels. In one example, the kit may include five (5) vessels, with the first starting at a nicotine strength of 100%, i.e., that matches the nicotine strength of the user's current vaping products, and the last ending at 0% or zero nicotine. In such an example, the nicotine strength would be about 100%, about 75%, about 50%, about 25%, and about 0% from the first to the last of the five vessels. In a similar example, the first vessel may have a nicotine strength of 90% of the user's current vaping products and the last vessel may have a nicotine strength of 10%. Thus, the five vessels in such an example would have gradually decreasing nicotine strengths of about 90%, about 70%, about 50%, about 30%, and about 10% from the first to the last vessel in such a kit.

In another example, the kit may include eleven (11) vessels gradually decreasing in nicotine strength by 10% per vessel from 100% to no nicotine. In other examples, the number of vessels in a kit may be from four (4) to ten (10) with the gradual nicotine strength decrease being 10% or more from vessel to vessel. In still other examples, the number of vessels in a kit may be eleven (11) or more, such as up to 20 vessels in one example. With fewer vessels in a kit, the nicotine strength drop from vessel to vessel will be larger, though the kit costs may be cheaper, and the cessation process may be quicker. However, at some point the strength drop at each step may be too severe for some users, such that a user will then experience withdrawal symptoms or return to a nicotine addiction. With more vessels in a kit, the nicotine strength drop from vessel to vessel will be smaller, though the kit costs may be higher, and the cessation process may take longer. However, at some point the strength drop at each step may be too small resulting in very little progression at each step, rendering the kit unnecessarily costly and the cessation process unnecessarily long.

In still other examples, the gradual decrease in nicotine strength through the vessels of the kit may be smaller at the higher strengths and greater at the lower strengths, instead of the consistent gradual reduction described above. This may be beneficial to users of the disclosed kits. This is because a reduction from the highest or a higher nicotine strength to the next lower nicotine strength may affect a user more and/or be more noticeable to the user than would a larger reduction from a lower nicotine strength to the next lower or to the lowest nicotine strength. Thus, in one example, the nicotine strength reduction from the first vessel or e-cigarette 22A to the last vessel or e-cigarette 22K may also gradually become larger or increase. In one example, the first nicotine reduction step may be on the order of 5% or 10% whereas the final nicotine reduction step may be on the order of 20%, 25% or even higher, and may end at zero nicotine for the final step.

As shown in FIG. 1 each of the vessels 22 of the kit may be marked with readily visible and sequential indicia. The sequential indicia can be marked on or applied to each of the plurality of vessels 22 of the kit 20, one per vessel. The indicia can be sequential in nature to make it easy for a user to determine the proper order of use of the vessels of the kit in order to properly follow the disclosed cessation program or method.

In one example, as shown in FIG. 1, the indicia may simply be numerical or alphanumeric indicia, such as the numbers ‘1’-‘10’ assigned and applied respectively to each of the e-cigarettes 22A-22K in the kit 20. As shown in FIG. 1, the first, highest nicotine strength e-cigarette 22A includes the number ‘1’ and the final or last, lowest nicotine strength e-cigarette 22K includes the number ‘10’. The remaining e-cigarettes 22B-22J are sequentially and respectively marked with the indicia ‘2’-‘9’ in this example. Thus, a user can easily select the vessel marked number ‘1’ to start the program and use the vessels in order from ‘1’ through ‘10’ to complete the program.

In other examples, the indicia may count down instead of counting up such that, for the kit 20, the user may start a first vessel marked ‘10’ having the highest nicotine strength and finish with a last vessel ‘1’ having the lowest nicotine strength. Also, the indicia need not be the numbers ‘1’-‘N’ but instead can be other meaningful alphanumeric indicia, various colors, different shapes, or other types of indicia, or combinations of any such indicia, within the spirit and scope of the present disclosure.

The volume of e-liquid or the substance containing nicotine held in one of the vessels, such as the vessels 22 in the above example, can also vary. In the described examples, the e-liquid volume can depend on the parameters of each step required to successfully move a user from a higher nicotine strength to the next lower nicotine strength during the program. The parameters can relate to the desired duration of each step, the amount of vapor that needs to be inhaled by a user for each step, and the like. To add more volume, the vessels can be increased in size to store or hold more e-liquid. Instead, two or more vessels at each step can instead be provided holding e-liquid of the same nicotine strength. In other words, two, three, or more of each of the vessels 22A, 22B, 22C, etc. in the above example may be provided, each holding the same nicotine strength e-liquid. The user would then consume or vape the e-liquid of those multiple vessels at each step before moving on to the next level of the highest remaining or next lower nicotine strength vessels.

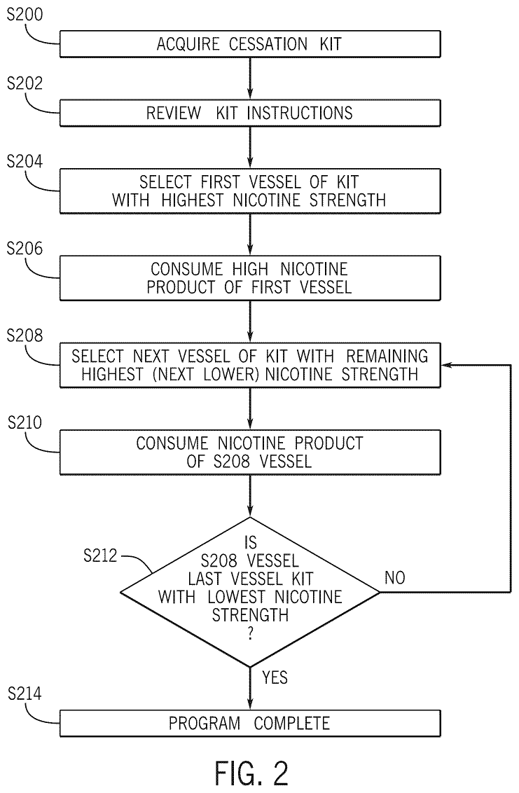

FIG. 2 shows one example of a nicotine cessation method according to the teachings of the present disclosure and using the kit 20 disclosed in FIG. 1 and described above. As should become evident to those of ordinary skill in the art upon reading the following description, the method may be modified in various ways without departing from the scope and spirit of the present disclosure.

With reference to FIGS. 1 and 2, from the viewpoint of a user of the kit 20, the method may include a step S200 of the user acquiring a nicotine cessation kit, such as a kit 20 as described above. The method may also include a step S202 of reviewing instructions provided with the acquired kit, such as the kit 20. The method may further include a step S204 of the user selecting the first or highest nicotine strength vessel, such as the e-cigarette 22A of the kit 20, to begin the nicotine cessation program or process. The user may readily identify the first e-cigarette 22A, from among the plurality of vessels 22 of the kit 20, by noting the indicia ‘1’ on the vessel.

The method may then include a step S206 of the user consuming, i.e., vaping, the contents of the first vessel or e-cigarette 22A to completion. Completing the first vessel or e-cigarette 22A, as well as the remaining vessels of the kit, may involve one or more of i) a prescribed period of time for vaping the contents of the first vessel, ii) a prescribed number of uses, i.e., vapes or hits or days from the first vessel, iii) vaping the entire e-liquid contents of the first vessel, or any combination thereof. In some examples, it may take one to two weeks at each step to gradually stepdown the user's nicotine dependency. It may also take about 25 milliliters (ml) per week, in one example, of e-liquid to accomplish each stepdown. The one to two week period allows the user's body sufficient time to adjust to a new nicotine level with each stepdown so the user may vape at their normal rate (i.e., puffs per day) when it is time to step down again to the next lower nicotine strength.

The method may include a step S208 of the user selecting the next vessel having the remaining highest (i.e., next lower) nicotine strength, such as the e-cigarette 22B of the kit 20. The user again may readily identify the next e-cigarette 22B, from among the plurality of vessels 22 of the kit 20, by noting the indicia ‘2’ on the vessel. The method may then include a step S210 of the user consuming, i.e., vaping, the contents of the second vessel or e-cigarette 22B to completion according to the aforementioned parameters (i), (ii), (iii), or any combination thereof.

For the kit 20, the user would repeat the steps of selecting the next remaining vessel with the highest remaining (i.e., next lower) nicotine strength of the plurality of vessels and consuming the contents to completion. According to the method of FIG. 2, the method may include a step S212 of determining whether the previously selected vessel is the final or last vessel having the lowest nicotine strength. If the selected vessel is the final or last vessel, such as e-cigarette 22K of the kit 20, by noting the appropriate indicia, i.e., the number ‘10’ on the vessel in this example (YES at S212), the method is then considered complete at step S214. If the selected vessel is not the last or final vessel of the kit, i.e., is not the e-cigarette 22K numbered ‘10’ of the kit 20 in this example (NO at S212), the user repeats steps S208 and S210 until completing the cessation program or method. Here, the user repeats steps S208 and S210 for each of the e-cigarettes 22C (number ‘3’), 22D (number ‘4’), 22E (number ‘5’), 22F (number ‘6’), 22G (number ‘7’), 22H (number ‘8’), 22J (number ‘9’), and finally, 22K (number ‘10’) to complete the program or method.

In one example, though not shown herein, a method according to the teachings of the present disclosure may include, from the viewpoint of a manufacturer or distributor, the steps of providing or manufacturing a kit, such as the kit 20, providing use instructions for the kit, and selling the kit direct to a user or indirectly to a distributor. The cessation program or method would then be carried out by a user, using the kit and instructions obtained thereby, according to the disclosed steps of the program or method.

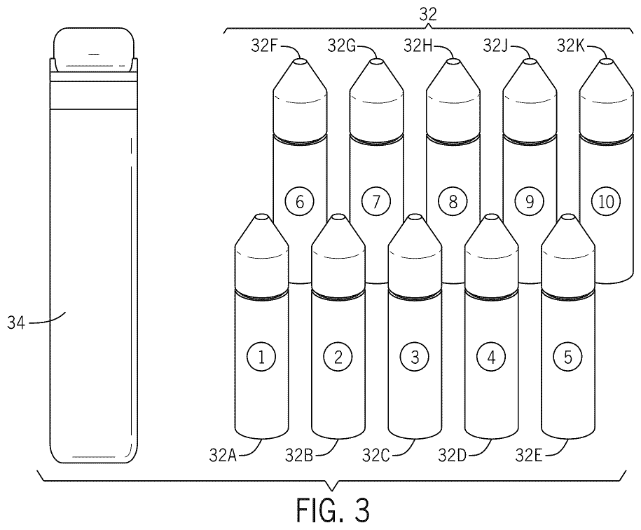

As noted above, various examples and embodiments of kits and methods for nicotine cessation are disclosed and described herein with reference to the drawings. FIG. 3 shows one example of a nicotine cessation kit 30, i.e., hereinafter the kit 30, that has been devised in accordance with the principles of the present disclosure. As should become evident upon reading the following description, the kit 30 may be modified in various ways without departing from the scope and spirit of the present disclosure.

The kit 30 in one example includes a plurality of vessels, where the plurality of vessels is generally identified as the vessels 32, and where the vessels 32 are configured to hold e-liquid. In this example, the vessels 32 are not configured to produce and deliver the inhalable vapor yielded from the e-liquid to the user. Thus, in this example, the vessels 32 may be bottles or other such containers, which are generally known in the art. The bottles 32 can hold e-liquid, which can be used simply to fill e-cigarettes or vape pens. In one example, the kit 30 may be provided with a re-usable e-cigarette 34 for the convenience of the user and configured to be suitable for and compatible with the e-liquid held in the bottles 32. In another example, the kit 30 can be provided only with the plurality of vessels, i.e., the bottles 32, and the user can be responsible for obtaining the appropriate e-cigarette(s). The configuration of the plurality of vessels, i.e., the bottles 32 in this example can again vary in contour design, color, size, form, closure design, dispensing element design, and the like without departing from the spirit and scope of the present disclosure. Each of the vessels 32 has a storage space or cavity (not shown) configured to hold a predetermined volume of the e-liquid. The amount of e-liquid that can be held by each of the vessels 32 can also vary depending on a number of variables that are discussed herein.

Referring to FIG. 3, the plurality of vessels 32 in this example may include a predetermined number of containers or bottles, such as bottles 32A-32K. As discussed above, the number of vessels 32 in the kit 30 may vary according to the needs of a particular user and/or according to a particular kit implementation. In this particular example, the plurality of vessels 32 of the kit 30 again includes ten (10) bottles 32A-32K.

In one example, each of the bottles 32A-32K may be preloaded or pre-filled with a predetermined volume of e-liquid. The volume of e-liquid preloaded into each of the bottles 32 may vary, as discussed below. Also, the nicotine amount or content, i.e., strength, of the e-liquid in each of the bottles 32A-32K in this example is different or varied in comparison to the remaining bottles of the kit 30, also as discussed above. In other words, each bottle 32A-32K of the plurality of vessels 32 is pre-filled with e-liquid having a predetermined nicotine strength. The strength or amount of nicotine is again designed to gradually decrease from a first vessel or bottle 32A, to among second through ninth vessels or bottles 32B-32J, and to a last or final vessel, i.e., a tenth bottle 32K in this example. Thus, the first bottle 32A in this example has the highest nicotine strength and the last or tenth bottle 32K has the lowest nicotine strength. The nicotine strength, gradual reduction, and e-liquid variables discussed above with respect to the kit 20 and the e-cigarettes 22A-22K is equally applicable to the kit 30 and the plurality of bottles 32, and thus is not described again here. The variable number of vessels discussed above with respect to the e-cigarettes 22 of the kit 20 is also equally applicable to the number of bottles 32 of the kit 30 in this example, and is thus also not described again here.

As shown in FIG. 3 each of the vessels, i.e., bottles 32, of the kit 30 may again be marked with readily visible and sequential indicia. The above discussion of the sequential indicia, variations, and options with respect to the e-cigarettes 22A-22K is also equally applicable to the bottles 32A-32K of the kit 30 in this example, and thus is not repeated here. As shown in FIG. 3, the first, highest nicotine strength bottle 32A again includes the number ‘1’ and the final or last, lowest nicotine strength bottle 32K includes the number ‘10’. The remaining bottles 32B-32J are sequentially and respectively marked with the indicia ‘2’-‘9’ in this example. Thus, a user can easily select the vessel, i.e., bottle marked number ‘1’ to start the program and use the vessels or bottles in order from ‘1’ through ‘10’ to complete the program.

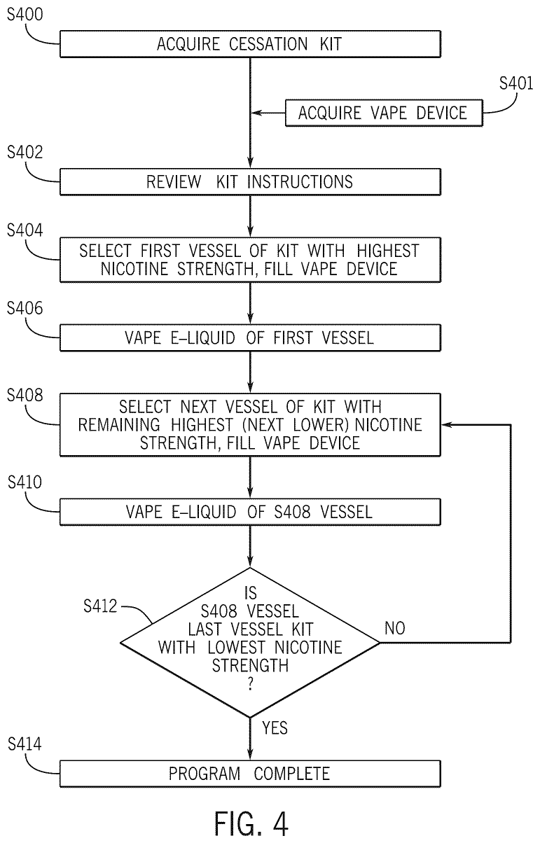

FIG. 4 shows one example of a nicotine cessation method according to the teachings of the present disclosure and using the kit 30 disclosed in FIG. 3 and described above. As should become evident to those of ordinary skill in the art upon reading the following description, the method may be modified in various ways without departing from the scope and spirit of the present disclosure.

With reference to FIGS. 3 and 4, from the viewpoint of a user of the kit 30, the method may include a step S400 of the user acquiring a nicotine cessation kit, such as a kit 30 as described above. The method may further include a step S401 of the user acquiring a vape device, i.e., the e-cigarette 34 to produce the vapor from the e-liquid for inhaling by the user. The step S401 may be a separate step or may be eliminated if the e-cigarette or vape device is provided as a part of the kit 30. The method may also include a step S402 of reviewing instructions provided with the acquired kit. In this example, and in the earlier example of the kit 20, instructions may be provided directly on the packaging for the kit, as a separate printed paper sheet along with the kit, and/or electronically via web access using a link provided to the user.

The method may further include a step S404 of the user selecting the first or highest nicotine strength vessel, such as the bottle 32A of the kit 30, and filling the e-cigarette 34 to begin the nicotine cessation program or process. The user may readily identify the first bottle 32A, from among the plurality of vessels 32 of the kit 30, by noting the indicia ‘1’ on the bottle.

The method may then include a step S406 of the user consuming, i.e., vaping, the contents of the first vessel or bottle 32A to completion using the e-cigarette 34. Completing the first vessel or bottle 32A, as well as the remaining vessels of the kit 30, may involve one or more of i) a prescribed period of time for vaping the contents of the first vessel, ii) a prescribed number of uses, i.e., vapes or hits or days from the first vessel, iii) vaping the entire e-liquid contents of the first vessel, or any combination thereof.

The method may include a step S408 of the user selecting the next vessel having the remaining highest (i.e., next lower) nicotine strength, such as the bottle 32B of the kit 30 and again filling the e-cigarette 34. The user again may readily identify the next bottle 32B, from among the plurality of vessels 32 of the kit 30, by noting the indicia ‘2’ on the bottle. The method may then include a step S410 of the user consuming, i.e., vaping, the contents of the second vessel or bottle 32B to completion according to the aforementioned parameters (i), (ii), (iii), or any combination thereof.

For the kit 30, the user would repeat the steps of selecting the next remaining vessel or bottle with the highest remaining (i.e., next lower) nicotine strength of the plurality of vessels and consuming the contents to completion. According to the method of FIG. 4, the method may include a step S412 of determining whether the previously selected vessel or bottle is the final or last vessel or bottle having the lowest nicotine strength. If the selected vessel or bottle is the final or last vessel, such as bottle 32K of the kit 30, by noting the appropriate indicia, i.e., the number ‘10’ on the vessel in this example (YES at S412), the method is then considered complete at step S414. If the selected vessel is not the last or final vessel of the kit, i.e., is not the bottle 32K numbered ‘10’ of the kit 30 in this example (NO at S412), the user repeats steps S408 and S410 until completing the cessation program or method. Here, the user repeats steps S408 and S410 for each of the bottles 32C (number ‘3’), 32D (number ‘4’), 32E (number ‘5’), 32F (number ‘6’), 32G (number ‘7’), 32H (number ‘8’), 32J (number ‘9’), and finally, 32K (number ‘10’) to complete the program or method.

The e-liquid volume for the vessels of the above-described kit examples and for completing each step for the above described methods may vary from as little as 10 ml to 200 ml. In one example, the vessels 22 and/or 32 may each be configured to hold about 25 ml to 50 ml of the e-liquid. In one particular example, the volume of e-liquid held by each vessel may be about 25 ml.

In one example, though not shown herein, a method according to the teachings of the present disclosure may include, from the viewpoint of a manufacturer or distributor, the steps of providing or manufacturing a kit, such as the kit 30, with or without the e-cigarette or vape device 34, providing use instructions for the kit, and selling the kit direct to a user or indirectly to a distributor. The cessation program or method would then be carried out by a user, using the kit and instructions obtained thereby, according to the disclosed steps of the program or method.

According to the teachings of the present disclosure, the “Vape Quit Kit” is designed to provide a user-friendly and systematic approach to nicotine cessation. The process may unfold as described above, and as follows, using the kit 20 as described above. The disclosed embodiments of a “Vape Quit Kit” offer a systematic and user-friendly approach to nicotine cessation. In these embodiments, the kit consists of ten disposable vape devices or ten e-liquid bottles, each uniquely numbered from ‘1’ to ‘10’, and pre-filled with e-liquids of varying nicotine strengths. The goal of these embodiments is to guide users through a ten-step program with gradual and uniform reductions in nicotine intake, ensuring a comfortable and effective transition to a nicotine-free state.

Step 1—Highest Nicotine Strength (e.g., 24 mg)—Users commence their journey with a disposable vape device numbered ‘1’, which is pre-filled with the highest nicotine strength e-liquid. This step serves as the starting point for the disclosed program or method.

Step 2—Gradual Reduction—After completing Step 1, users progress to the disposable vape device numbered ‘2’. Each successive step features a gradual reduction in nicotine strength that may be uniform in proportion. This ensures a smooth and consistent transition between the various steps of the program or method.

Step 3 to Step N-1—Incremental Reduction—Users continue through the program in this manner, proceeding to the next disposable vape device(s) numbered ‘3’ through ‘N-1’ with each step, where ‘N’ is the number of e-liquid vessels, i.e., the e-cigarettes 22 of the kit. At every step, users experience a proportionate decrease in nicotine strength. This incremental reduction minimizes the disparity between nicotine levels and reduces discomfort and withdrawal symptoms. For the kit 20, these steps include steps ‘3’-‘9’.

Step N—Lowest Nicotine Strength (e.g., 0 mg)—The final step of the program features a disposable vape device pre-filled with nicotine-free e-liquid, marking the attainment of a nicotine-free state. This would include the vape device e-cigarette numbered ‘10’ for the above described kit 20.

The e-liquid volume to be consumed or inhaled via vaping may be varied, as noted above. The volume may depend on how long a user is to remain under each step of the program and/or how many hits a particular vessel is intended to provide to a user according to the above described methods.

To overcome the above-noted problems with prior known nicotine cessation techniques, the inventor has implemented a more gradual and user-friendly multi-step program or method. The disclosed approach ensures that individuals experience minimal disparity in nicotine levels between each step. This significantly reduces discomfort and withdrawal symptoms. The ultimate objective of the present disclosure is to enable a smooth transition to zero nicotine intake, offering a more effective and less challenging method for individuals looking to quit vaping and overcome the associated nicotine dependency.

The disclosed nicotine cessation kits comprise a variable number of vape devices or e-liquid bottles, i.e., vessels. Each may be uniquely numbered and thoughtfully pre-filled with e-liquid having varying nicotine strengths. This progression enables users to experience a gradual reduction in nicotine intake until they reach nicotine-free status. The disclosed kits are designed to facilitate a user-friendly and seamless process for those seeking to reduce their nicotine dependency.

The kits include disposable electronic vape devices or bottles for filling a vape device, each individually numbered for easy reference. These disposables or bottles serve as each kit's central components, and their design is integral to each kit's functionality. Pre-filled with e-liquid, each disposable or bottle corresponds to a specific nicotine strength level, allowing users to progressively reduce their nicotine intake.

In one example, users may commence their journey with a disposable vape device or a bottle, pre-filled with the highest nicotine strength (e.g., 24 mg). Users may then proceed sequentially to lower-numbered disposables or bottles when they feel prepared to reduce their nicotine intake. Thus, the user may take a different journey than other users while using the disclosed kits and methods.

In contrast to the known, abrupt, half-step reduction method, the disclosed kits utilize a multi-step, consistent program or method. Nicotine strength decreases gradually with each disposable or bottle, ensuring users experience minimal variation between steps, reducing discomfort and withdrawal symptoms.

As users continue to advance through the disposables or the bottles of the disclosed kits, the users gradually decrease their nicotine intake without experiencing unpleasant symptoms caused by abrupt changes in nicotine strength. The disposables and bottles of the disclosed kits are designed for ease of use, ensuring a smooth transition for users.

The disposable vape devices may be ergonomically designed, lightweight, and user-friendly. They may feature either a button or a draw-activated mechanism for ease of use. The numbering system or other indicia may be prominently displayed on each disposable or bottle, easily and clearly guiding users along their nicotine cessation journey. The e-liquids are securely contained within the vessels, i.e., the bottles or the vape devices or e-cigarettes.

The disclosed kits and methods are designed to offer a seamless transition for users. One of the kits' most notable features are their ability to create a seamless transition from one nicotine strength to the next, making it easier for users to manage their cravings and adapt to lower nicotine levels. The kits may be engineered with the user's comfort and success in mind, minimizing the challenges often associated with nicotine reduction. By offering a full range of nicotine strengths and a gradual progression, the kits serve as a comprehensive solution for individuals looking to quit vaping. One of the kits provides the added convenience of disposable vape devices. Both kits may be configured to allow the user to set the starting nicotine strength of the first vessels, thus allowing the users to tailor the program or method to their specific nicotine situation.

Although certain nicotine cessation kits, systems, methods, and stepdown programs have been disclosed and described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A kit for nicotine cessation, the kit comprising:
a plurality of vessels each holding a substance for consumption by a user;
a first vessel of the plurality of vessels with the substance having a highest nicotine content;
a last vessel of the plurality of vessels with the substance having a lowest nicotine content;
a second vessel of the plurality of vessels with the substance having a second nicotine content lower than the highest nicotine content; and
at least a third vessel of the plurality of vessels with the substance having a third nicotine content higher than the lowest nicotine content and lower than the second nicotine content,
wherein nicotine content decreases gradually from the highest nicotine content to the lowest nicotine content, and
wherein the substance held by the plurality of vessels is configured to be consumed in order from the first vessel to the last vessel.

2. The kit of claim 1, wherein the substance is an e-liquid configured to be consumed using an e-cigarette.

3. The kit of claim 1, wherein each of the plurality of vessels is an e-cigarette filled with the substance, and wherein the substance is an e-liquid configured to be consumed through vaping.

4. The kit of claim 3, wherein each e-cigarette is a disposable e-cigarette pre-filled with the e-liquid.

5. The kit of claim 1, wherein each of the plurality of vessels is a fluid container holding the substance, and wherein the substance is an e-liquid configured to be consumed through vaping.

6. The kit of claim 5, further comprising an e-cigarette provided as a part of the kit for vaping the e-liquid.

7. The kit of claim 1, wherein the plurality of vessels includes unique indicia, and wherein the unique indicia inform the user which of the plurality of vessels is the first vessel, the second vessel, at least the third vessel, and the last vessel.

8. The kit of claim 1, wherein each vessel of the plurality of vessels includes a visible numerical indicia so that the plurality of vessels is sequentially numbered from the first vessel to the last vessel in the order to be consumed by the user.

9. The kit of claim 1, wherein each vessel of the plurality of vessels is a nicotine pouch, a nicotine patch, or an oral consumable.

10. The kit of claim 1, wherein the plurality of vessels includes at least ten vessels inclusive of the first, second, third, and last vessels.

11. The kit of claim 10, wherein the at least ten vessels include exactly ten vessels, and wherein the ten vessels are provided with sequential visible indicia as numbers from the first vessel bearing a ‘1’ to the last vessel bearing a ‘10’.

12. The kit of claim 1, wherein the lowest nicotine content of the last vessel is in a range from 25% to 0% of an initial nicotine intake of the user.

13. The kit of claim 1, wherein the highest nicotine content of the first vessel is in a range from 100% to 75% of an initial nicotine intake of the user.

14. A method for nicotine cessation, the method comprising:
acquiring a nicotine cessation kit including a plurality of vessels with a substance for consumption by a user, wherein a first vessel of the plurality of vessels includes the substance having a highest nicotine content, and a last vessel of the plurality of vessels includes the substance having a lowest nicotine content;
selecting a first vessel of the plurality of vessels with the substance having a highest nicotine content;
consuming the substance of the first vessel;
selecting a next vessel of the plurality of vessels having a highest remaining nicotine content;
consuming the substance of the next vessel;
repeating selecting a next remaining vessel and consuming the substance of the next remaining vessel until only the last vessel remains; and
consuming the substance of the last vessel,
wherein nicotine content decreases gradually from the highest nicotine content to the lowest nicotine content.

15. The method of claim 14, wherein the substance is an e-liquid and the steps of consuming include vaping the e-liquid using an e-cigarette.

16. The method of claim 15, wherein each of the plurality of vessels is a disposable e-cigarette pre-filled with the e-liquid, and wherein the method includes discarding each disposable e-cigarette when the e-liquid therein has been consumed through vaping.

17. The method kit of claim 15, wherein each of the plurality of vessels is a container holding the e-liquid, and wherein the method includes filling the e-cigarette with the e-liquid from the containers and consuming the e-liquid through vaping.

18. The method of claim 14, wherein the plurality of vessels includes sequentially numbered visible indicia, and wherein the steps of selecting include identifying the first vessel by the visible indicia ‘1’, and identifying each next vessel in sequential numbered order to the last vessel.

19. The method of claim 14, wherein each vessel of the plurality of vessels is a nicotine pouch, a nicotine patch, or an oral consumable.

20. The method of claim 14, wherein the plurality of vessels includes at least ten vessels inclusive of the first, each next, and the last vessels.
